(19) United States
(12) Patent Application Publication
(10) Pub. No.: US 2022/0133232 A1
(43) Pub. Date: May 5, 2022

(54) Electronic Skin

(71) Applicant: AI Biomaterial Healthtech Ltd., Kwun Tong KLN (HK)

(72) Inventor: Yen-Yi Ho, Taoyuan City (TW)

(21) Appl. No.: 17/147,561

(22) Filed: Jan. 13, 2021

(30) Foreign Application Priority Data
Nov. 3, 2020 (TW) 109138272

(51) Int. Cl. A61B 5/00

(52) U.S. Cl. CPC A61B 5/6832; A61B 5/7445; A61B 2562/164; A61B 2560/0214; A61B 5/0015

(57) ABSTRACT

An electronic skin for attaching on a user's skin and detecting the user's physiological information is disclosed. The electronic skin includes a pasting layer, an electronic unit layer and a covering layer. The electronic unit layer includes a transmission module, a physiological information sensor, a using state sensor, a storage module, a processor, a power module and a flexible substrate. The physiological information sensor is used for sensing the physiological information. The using state sensor is used for sensing the using state of the electronic skin. The flexible substrate is located on the pasting layer and under the transmission module, the physiological information sensor, the using state sensor, the storage module, the processor and the power module. The covering layer is located on the electronic unit layer.

### BACKGROUND OF THE INVENTION

### 1. Field of the Invention

The present invention relates generally to an electronic skin, and more particularly to an electronic skin for attaching on a user's skin and detecting physiological information of the user.

### 2. Description of the Related Art

A patient suffering from an infectious disease will usually stay in an isolation ward to prevent spread of the bacteria or virus causing the infectious disease. Nurses must wear such protective equipment as a mask, an isolation gown, or a face shield when entering the isolation ward to examine the patient, so as to avoid infection.

However, because wearing complete protective equipment is very time-consuming, the nurse in charge of the isolation ward will only take time to wear protective equipment during specifically planned period before entering the isolation ward to examine the patient. Therefore, the nurse cannot enter the isolation ward to review the physiological information of the patient at any time. Therefore, it is necessary to provide a device that facilitates detection of the user's physiological information at any time.

### SUMMARY OF THE INVENTION

It is an object of the present invention to provide an electronic skin for attaching on a user's skin and for detecting the user's physiological information.

To achieve the abovementioned objects, an electronic skin according to the present invention is used for attaching on a user's skin and for detecting the user's physiological information. The electronic skin includes a pasting layer, an electronic unit layer and a covering layer. The electronic unit layer includes a transmission module, a physiological information sensor, a using state sensor, a storage module, a processor, a power module and a flexible substrate. The physiological information sensor is used for sensing the user's physiological information. The using state sensor is used for sensing the using state of the electronic skin. The power module is electrically connected to the transmission module, physiological information sensor, using state sensor, storage module and processor. The flexible substrate is located on the pasting layer, and under the transmission module, physiological information sensor, using state sensor, storage module, processor and power module. The covering layer is located on the electronic unit layer.

According to an embodiment of the present invention, wherein said electronic unit layer further includes a block chain module, and said block chain module is electrically connected to the power module and a block chain network.

According to an embodiment of the present invention, wherein said electronic unit layer further includes a display module, and said display module is electrically connected to said power module.

According to an embodiment of the present invention, wherein said covering layer further includes a hole, and said hole is located on the display module.

According to an embodiment of the present invention, wherein said transmission module further includes an antenna, and said antenna is used for enhancing the transmission signal of said transmission module.

According to an embodiment of the present invention, wherein said power module is a thin-film solar cell, and said thin-film solar cell obtains electric power through reception of sun light.

According to an embodiment of the present invention, wherein said pasting layer is made of silicone gel.

According to an embodiment of the present invention, wherein said flexible substrate is made of silicone rubber.

According to an embodiment of the present invention, wherein said covering layer is made of polyurethane (PU).

### BRIEF DESCRIPTION OF THE DRAWINGS

FIG. 1 is a schematic diagram showing an electronic skin in an embodiment of the present invention attached on a user's skin.

FIG. 2 is a schematic diagram of an electronic skin in an embodiment of the present invention.

FIG. 3 is a sectional view of an electronic skin in an embodiment of the present invention along the section line AA in FIG. 1.

FIG. 4 is a system architecture diagram of an electronic skin in an embodiment of the present invention.

### DETAILED DESCRIPTION OF THE PREFERRED EMBODIMENT

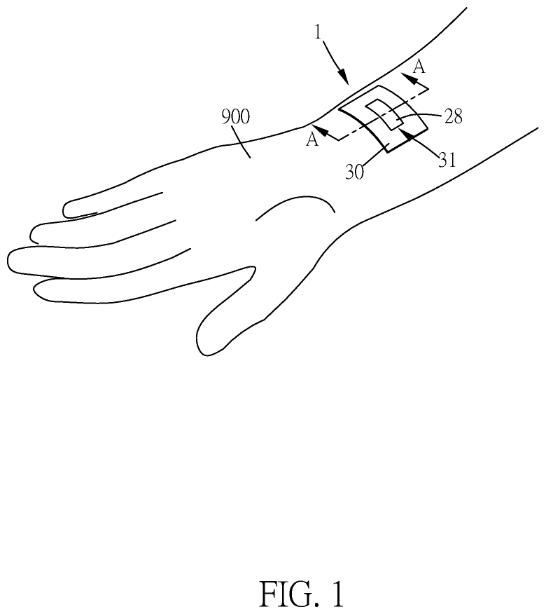

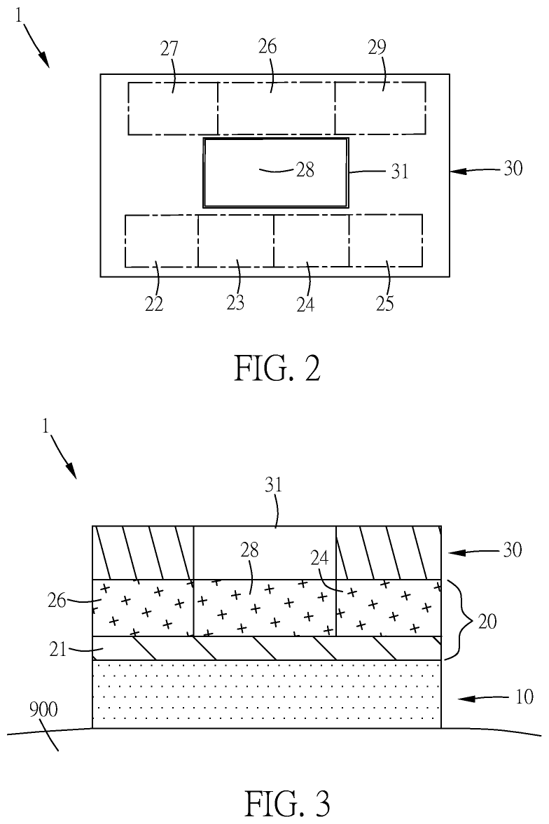

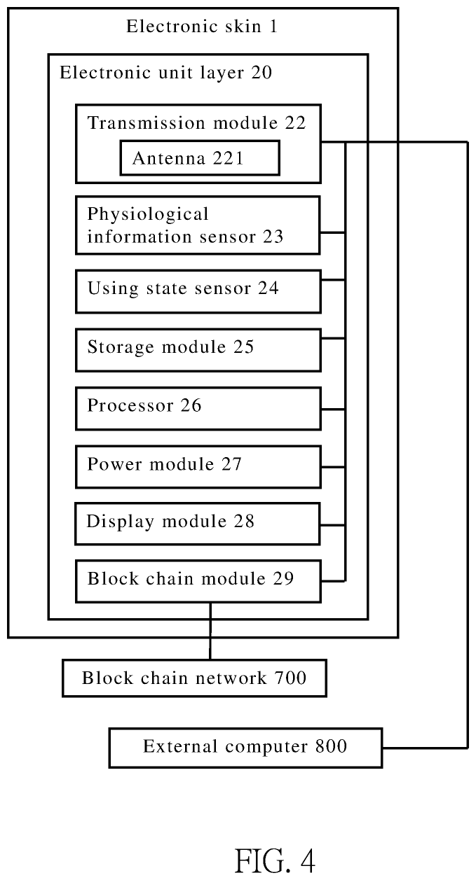

Referring to FIG. 1 to FIG. 4, which show an electronic skin in an embodiment of the present invention, FIG. 1 is a schematic diagram showing an electronic skin in an embodiment of the present invention attached on a user's skin; FIG. 2 is a schematic diagram of an electronic skin in an embodiment of the present invention; FIG. 3 is a sectional view of an electronic skin in an embodiment of the present invention along the section line AA in FIG. 1 is a sectional view of an electronic skin in an embodiment of the present invention along the section line AA in FIG. 1; FIG. 4 is a system architecture diagram of an electronic skin in an embodiment of the present invention.

Referring to FIG. 1 to FIG. 3, in the embodiment of the present invention, the electronic skin 1 is for attaching on a user's skin 900, and for detecting the user's physiological information at any time. The electronic skin 1 includes a pasting layer 10, an electronic unit layer 20 and a covering layer 30.

In the embodiment of the present invention, the pasting layer 10 is used for attaching on the skin 900, and for separating the electronic unit layer 20 from the skin 900, so as to prevent the electronic unit layer 20 from directly contacting the skin 900 to cause stimulation. The pasting layer 10 is made of a flexible and elastic material that will unlikely cause allergy of the skin, for example, silicone gel. Thus, it will not stimulate the skin on which it is attached.

Referring to FIG. 2 to FIG. 4, in the embodiment of the present invention, the electronic unit layer 20 includes a flexible substrate 21, a transmission module 22, a physiological information sensor 23, a using state sensor 24, a storage module 25, a processor 26, a power module 27, a display module 28 and a block chain module 29. The flexible substrate 21 is made of a flexible material, such as silicone rubber, and is located on the pasting layer 10 and under the transmission module 22, physiological information sensor 23, using state sensor 24, storage module 25, storage module 25, processor 26 and power module 27. The flexible substrate 21 can bend slightly to match the surface of the skin 900, so that the electronic skin 1 can be stably attached on the skin 900.

The transmission module 22 (such as a network card) is used for transmitting data to an external computer 800, and the transmission module 22 can also be accessed by an external computer 800 to control the electronic skin 1; the transmission module 22 further includes an antenna 221 and the antenna 221 is used for enhancing the transmission signal of the transmission module 22. The physiological information sensor 23 is a chip with the function to sense the physiological information, and is used for sensing the user's physiological information. Physiological information includes but is not limited to body temperature, heartbeat, and blood pressure. The using state sensor 24 is a compound chip set with the function to sense the using state of the electronic skin 1, having a global positioning system chip and a temperature sensing chip. The using state sensor 24 is used for sensing the using state of the electronic skin 1 and check if it is normal. Using state includes but is not limited to the current location and current temperature of the electronic skin 1.

The storage module 25 (such as a RAM) is used for storing the data of the physiological information and the using state. The processor 26 is used for coordinating and processing the operations of the electronic units. The power module 27 is a thin-film solar cell. The thin-film solar cell obtains electric power through reception of sun light, and supplies the electric power to other electronic units. The power module 27 is electrically connected to the transmission module 22, the physiological information sensor 23, the using state sensor 24, the storage module 25, the storage module 25 and the processor 26. The display module 28 can be an LED light. The display module 28 is electrically connected to the power module 27. The display module 28 can obtain electric power to emit light and provide information to the user in form of light. For example, the display module 28 may emit warning light when the physiological information sensor 23 or the using state sensor 24 detects abnormal information. The warning light can remind the user that the current physiological information or the current using state of the electronic skin 1 is abnormal and a medical professional shall be notified for assistance.

The block chain module 29 is a computing chip, and is electrically connected to the power module 27 and a block chain network 700. The block chain module 29 records the circulation history of the electronic skin 1 in a block chain network 700. Via the traceability and unlikelihood of manipulation ensured by the block chain, the circulation history of the electronic skin 1 can be recorded and tracked. According to the embodiment of the present invention, the circulation history of the electronic skin 1 includes the place of origin of the electronic skin 1, the organization using the electronic skin 1, and the storage and transportation process of the electronic skin 1. In addition, the circulation history of the electronic skin 1 can also be input through an external computer 800 into the block chain module 29 for record. The block chain module 29 will then record the circulation history of the electronic skin 1 in the block chain network 700. Thus, based on the traceability and unlikelihood of manipulation ensured by the block chain, the circulation history of the electronic skin 1 can be clearly recorded and managed.

In the embodiment of the present invention, the covering layer 30 is made of polyurethane (PU), and is located on the electronic unit layer 20. The covering layer 30 is used for protecting the electronic unit layer 20, to avoid exposure of the electronic unit layer 20 and possible damage caused by dust. The covering layer 30 includes a hole 31. The hole 31 is located on the display module 28. The hole 31 is used for exposing the light of the display module 28 for the user to see.

To use the electronic skin 1 of the present invention, the user can put the electronic skin 1 on the skin 900 of the wrist, and the pasting layer 10 will be attached on the skin 900, and separate the electronic unit layer 20 from the skin 900, preventing the electronic unit layer 20 from directly contacting the skin 900 to cause stimulation. As the pasting layer 10 is made of a material that will unlikely cause allergy of the skin, even if it is attached on the skin 900 for a long period of time, it will not cause stimulation of the skin 900 on which it is attached. When the electronic skin 1 is attached on the skin 900, the physiological information sensor 23 will continuously detect the user's physiological information (such as body temperature, heartbeat, and blood pressure); the using state sensor 24 will detect the using state of the electronic skin 1 and check if it is normal. For example, it will detect if the electronic skin 1 is located in the proper medical facility, or if the electronic skin 1 is under an appropriate temperature range, so as to avoid damage of the electronic skin 1 cause by overheat. The data of the physiological information and using state detected will be stored in the storage module 25, and the transmission module 22 can transmit the data stored in the storage module 25 to an external computer 800, so that a medical professional can use an external computer 800 to review the physiological information and using state detected by the electronic skin 1 whenever necessary. In addition, when the physiological information sensor 23 or the using state sensor 24 detects abnormal information, the display module 28 can emit warning light to remind the user that the current physiological information or using state of the electronic skin 1 is abnormal, and a medical professional shall be notified for assistance; or, when the physiological information sensor 23 or the using state sensor 24 detects abnormal information, the transmission module 22 can transmit the abnormal information to an external computer 800, so as to notify a medical professional for assistance.

In addition, the medical professional can also operate an external computer 800 to input the circulation history of the electronic skin 1 into the block chain module 29, and through the block chain module 29, the circulation history of the electronic skin 1 can be recorded in the block chain network 700. Based on the function of the block chain to trace the circulation history of an article, the relevant place of origin of the electronic skin 1, the organization using the electronic skin 1, the storage and transportation process of the electronic skin 1 can all be recorded in the block chain network 700. Thus, based on the traceability and unlikelihood of manipulation ensured by the block chain, the circulation history of the electronic skin 1 can be properly recorded and managed. Based on the function of the block chain to trace the circulation history of an article, a medical professional can also quickly track the identity of the user and the relevant place of usage or place of production of the electronic skin 1.

Based on the innovative design of the electronic skin 1 according to the present invention, the electronic skin 1 can be attached on a user's skin, to continuously detect the user's physiological information. If the electronic skin 1 detects abnormal physiological information, the display module 28 will emit warning light to remind the user that the current physiological information or the current using state of the electronic skin 1 is abnormal, and a medical professional shall be notified for assistance. Or, the transmission module can transmit the abnormal information to an external computer, so as to notify a medical professional for assistance. In addition, as the electronic skin 1 has the function to continuously detect the user's physiological information and to transmit data over long distance, it can be applied for remote medical care, or remote monitoring of the user's physiological information. Moreover, it can also be applied for long-term monitoring of the user's physical changes and for management of the physical cycles. For example, it can be used for recording body temperature changes to monitor women's menstruation and ovulation.

What is claimed is:

1. An electronic skin, for attaching on a user's skin and detecting a user's physiological information, the electronic skin including:
a pasting layer, for attaching on the user's skin;
an electronic unit layer, including:
a transmission module;
a physiological information sensor, for sensing the user's physiological information;
a using state sensor, for sensing a using state of the electronic skin;
a storage module;
a processor;
a power module, electrically connected to the transmission module, the physiological information sensor, the using state sensor, the storage module and the processor; and
a flexible substrate, located on the pasting layer, and under the transmission module, the physiological information sensor, the using state sensor, the storage module, the processor and the power module; and
a covering layer, located on the electronic unit layer.

2. The electronic skin defined in claim 1, wherein the electronic unit layer further includes a block chain module, and the block chain module is electrically connected to the power module and a block chain network.

3. The electronic skin defined in claim 2, wherein the electronic unit layer further includes a display module, and the display module is electrically connected to the power module.

4. The electronic skin defined in claim 3, wherein the covering layer further includes a hole, and the hole is located on the display module.

5. The electronic skin defined in claim 4, wherein the transmission module further includes an antenna, and the antenna is used for enhancing a transmission signal of the transmission module.

6. The electronic skin defined in claim 5, wherein the power module is a thin-film solar cell, and the thin-film solar cell obtains electric power through reception of sun light.

7. The electronic skin defined in claim 6, wherein the pasting layer is made of silicone gel.

8. The electronic skin defined in claim 7, wherein the flexible substrate is made of silicone rubber.

9. The electronic skin defined in claim 8, wherein the covering layer is made of polyurethane (PU).
